(19) United States
(12) Patent Application Publication
(10) Pub. No.: US 2022/0104639 A1
(43) Pub. Date: Apr. 7, 2022

(54) METHOD AND DEVICE FOR THE PREVENTION OF THE SPREAD OF A COMMUNICABLE DISEASE

(71) Applicant: Elizabeth M. Menendez, Kingston, NY (US)

(72) Inventor: Elizabeth M. Menendez, Kingston, NY (US)

(21) Appl. No.: 16/948,845

(22) Filed: Oct. 2, 2020

(51) Int. Cl. A47G 9/00; A61G 13/00; A61H 37/00

(52) U.S. Cl. CPC A47G 9/007; A61G 13/009; A61H 2201/01; A61H 2201/1607; A61H 37/00

(57) ABSTRACT

A device and method for preventing the spread of a communicable disease during a close proximity services session is provided. The method includes assembling a device to a face cradle of a massage table prior to the close proximity services session, wherein the device captures one or more respiratory droplets excreted by a client during the close proximity services session. The device includes a closed sidewall, an open sidewall, a top portion mechanically connected to the closed and to the open sidewall, wherein the top portion includes a securing means for securing the top portion to the face cradle and a bottom portion mechanically connected to the closed sidewall.

### BACKGROUND OF THE INVENTION

The popularity and widespread use of massage tables has risen in recent years. Massage tables are used not only for basic massage therapy but also for medical examinations, reflexology, physiotherapy, acupuncture, reiki healing, and the like. A typical table has an easily cleaned, padded surface, and a face cradle that allows the client to comfortably rest their face and breathe while lying face down. Unfortunately, for some people, breathing efficiently in this setting may be challenging.

The close proximity of the client to the therapist or healthcare professional performing services with a massage table creates an environment conducive to the spread of infection. With the pandemic conditions of the Coronavirus Disease 2019 (COVID-19), this proximity has become even more of an issue as the recommended distance between people to avoid spreading infection is six feet. Public health officials further recommend (and in some places mandate) that if social distancing of six feet is not possible, the mouth and nose should be covered with a cloth (or other material) face cover. Wearing a face cover while face down on a massage table may aggravate or create breathing discomfort and difficulties in some people.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 illustrates a typical close proximity services session 100 using a massage table.

FIG. 2 is a flowchart illustrating a method for prevention of the spread of communicable disease during close proximity services sessions such as illustrated in FIG. 1 in accordance with some embodiments.

FIG. 3 is a front perspective view illustrating a barrier device in accordance with some embodiments.

FIG. 4 is a rear perspective view illustrating a barrier device in accordance with some embodiments.

FIG. 5 is a side perspective view illustrating a barrier device attached to a face cradle of a massage table in accordance with some embodiments.

FIG. 6 is a side perspective view illustrating a barrier device for attachment underneath a massage table.

FIG. 7 is a flowchart illustrating a method of filtration virus-laden large droplets during close proximity services sessions in accordance with some embodiments.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION OF THE INVENTION

An aspect of the specification provides a method for preventing the spread of a communicable disease during a close proximity services session using a massage table incorporating a face cradle by assembling a device to the face cradle prior to the close proximity services session, where the device captures one or more respiratory droplets excreted by a client during the close proximity services session. The device includes a closed sidewall, an open sidewall, a top portion mechanically connected to the closed and to the open sidewall, wherein the top portion includes a securing means for securing the top portion to the face cradle and a bottom portion mechanically connected to the closed sidewall.

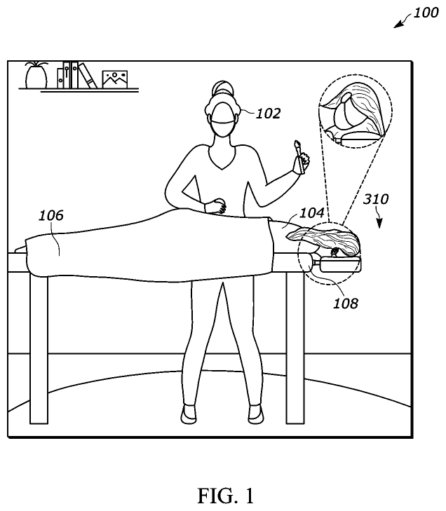

FIG. 1 illustrates a typical close proximity services session 100 using a massage table as is known in the art. As illustrated, a practitioner 102 provides various therapeutic or medical services to a client 104. The services, for example, may include massage therapy, medical examinations, reflexology, physiotherapy, acupuncture, reiki healing, and the like. The client 104 lies on a massage table 106, while the practitioner 102, in close proximity, performs such services. For many of the services, the client 104 lies face down on the massage table 106 with the client's face resting in a face cradle 310 near a front edge 108 of the massage table 106.

The close proximity of the practitioner 102 and the client 104 creates a potential for the spread of communicable diseases. Communicable diseases are transmitted from person to per-son primarily via virus-laden large droplets that are generated when infected persons cough or sneeze. These large droplets can then be directly deposited onto the mucosal surfaces of the upper respiratory tract of susceptible persons who are in close proximity (for example, within 6 feet) to the droplet source. In practice, therapy services performed by the practitioner 102 on the client 104 using the massage table 106 are close proximity services. There is no known practical alternative of performing such services from a safe distance. Transmission from the practitioner 102 to the client 104 or vice versa may occur through direct as well as indirect contact with infectious respiratory secretions or infectious expiratory droplets or airborne droplet nuclei. The Centers for Disease Control and Prevention (CDC) provide guidelines suggesting to wear a face covering to avoid transmission of infectious droplets. Further, face coverings in public are mandated in certain geographic locations due to high levels of communicable disease cases, such as COVID-19.

During traditional close proximity services sessions, breathing while lying face down is difficult due to accumulation of toxins in sinuses which ultimately may lead to respiratory conditions such as nasal congestion. Wearing a face covering may aggravate these respiratory issues.

During chronic respiratory conditions such as asthma or Chronic obstructive pulmonary disease (COPD), covering a client's mouth and nose may be especially challenging. The physical barrier of the face mask makes it harder to take in air; it also traps some carbon dioxide as one exhales, which means he/she ends up breathing in air that is warmer and moist. For some, these conditions may feel suffocating.

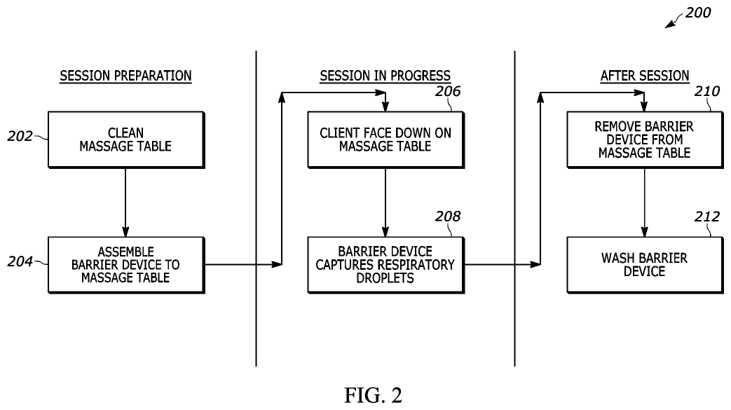

FIG. 2 is a flowchart illustrating a method 200 for prevention of the spread of communicable disease during close proximity services sessions such as illustrated in FIG. 1 in accordance with some embodiments.

A process of the prevention of the spread of communicable disease during close proximity services sessions will be described hereinafter with reference to FIG. 2 indicated as 200. The process begins with operation 202 for preparation of close proximity services session 100. In operation 202, the practitioner 102 may clean the massage table 106 as illustrated in FIG. 1. Next, in operation 204 the practitioner 102 assembles a device to the massage table 106. The device hereinafter may be referred to as a barrier device for clarity of explanation. The barrier device may be installed on a face cradle of the massage table 106 for the entire duration of the close proximity services session. After operation 202 and 204 the practitioner 102 may proceed with the close proximity services session 100 as illustrated in FIG. 1. It will be appreciated by those of ordinary skill in the art that the close proximity services session preparation may be performed by the practitioner, or any other qualified personnel and may be performed at any time before a close proximity services session begins in accordance with some embodiments.

In operation 206, during the progression of the close proximity services session the client 104 may lie face down on the massage table 106 with the client's face resting in the face cradle of the massage table 106 as illustrated in FIG. 1. Next, during operation 208, the barrier device acts as a collector to capture all respiratory droplets excreted from the client 104 during the close proximity services session 100. The operation 208 provides for the prevention of the spread of communicable diseases as will be described in further detail hereinafter. In operation 210, after each close proximity services session 100 the barrier device may be removed from the face cradle. In operation 212, the removed barrier device may be washed before its reuse. It will be appreciated by those of ordinary skill in the art that the method illustrated in FIG. 2 is for a single close proximity services session 100 for the client 104. In practice, the method would be repeated for each new close proximity services session 100 for each new client 104. In this manner, communicable diseases may be prevented while maintaining the practicality and manner of the therapy as well as adhering to the guidelines and/or mandates for social distancing and face masks.

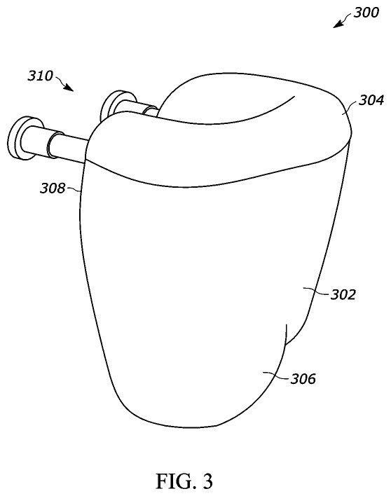

FIG. 3 is a front perspective view illustrating a barrier device 300 for use in the method 200 of FIG. 2 in accordance with some embodiments. In one embodiment, the barrier device 300 includes a closed sidewall 302, a top portion 304 of the closed sidewall 302, a bottom portion 306 of the closed sidewall 302 and a securing means 308 of the top portion 304.

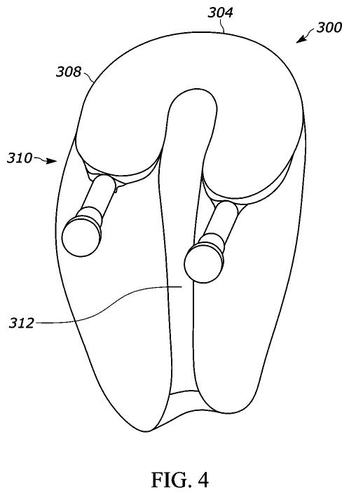

FIG. 4 is a rear perspective view of the barrier device 300 in accordance with some embodiments. In one embodiment the barrier device 300 includes an open sidewall 312, the top portion 304 of the open sidewall 312 which further includes the securing means 308 of the top portion 304 of the open sidewall 312.

Referring to FIGS. 3 and 4, the securing means 308 of the top portion 304 is configured to releasably retain the top portion 304 on the face cradle 310 of the massage table 106. Securing means may be configured to be attached along the top portion 304 to the closed sidewall 302 while providing easy access for removal of the barrier device 300. The top portion 304 mechanically connects to the closed sidewall 302 and to the open sidewall 312. The bottom 306 of the closed sidewall 302 mechanically connected to the closed sidewall 302 and form a vertically cylindrical hammock shape. It will be appreciated by those of ordinary skill in the art that in one embodiment the securing means comprises a permanent attachment such as a sewn, stapled, or other permanent attachment. It will further be appreciated that in alternative embodiments the securing means comprises a removable attachment such as a Velcro, snap, button, or other removable attachment. The securing means 308 is configured to par-tially receive the face cradle 310 and to retain the closed sidewall 302 on the face cradle 310. The securing means 308 further positioned the closed sidewall 302 and open sidewall 312 below the face cradle 310.

The securing means 308 may include hook-and-loop fasteners, or one or more snap closures, elastic elements such as rubber that will be appropriately fitted to the face cradle to facilitate easy removal and replacement of the barrier device on to a face cradle 300. The barrier device 300 that attaches to the face cradle 310 may be made of single or multilayer washable product materials such as cotton, polyester, copper fabric, quilting fabric and silk that is replaced after each use and may be easily cleaned and disinfected after each use.

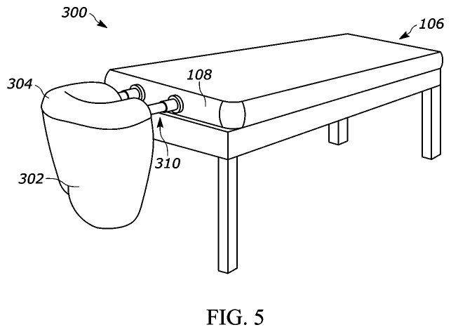

FIG. 5 is a side perspective view showing the barrier device 300 for attachment to the face cradle 310 of the massage table 106. In one embodiment the barrier device 300 attaches to the face cradle 310 and is installed onto the massage table 106. The barrier device 300 further includes the closed sidewall 302 and open sidewall 312 running vertically downwards to the massage table 106. The closed sidewall 302 is further positioned opposite to the open sidewall 312 to facilitate the comfort to the client while face down on the massage table 106. The closed sidewall 302 is positioned towards the surrounding massage therapy room and away from the front edge 108 of the massage table 106. The closed sidewall 302 by its positioning prevents the spread of respiratory droplets in the surrounding therapy room; thereby replacing the functionality of the face mask. The open side wall 312 is configured in close connection to the face cradle 310 and the front edge 108 of the massage table 106 which maintains sufficient air flow in the barrier device 300.

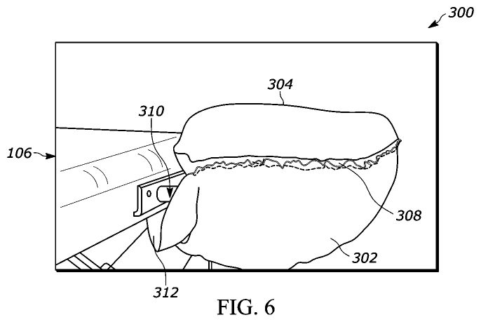

FIG. 6 is a side perspective view showing the open sidewall 312 of the barrier device 300 for attachment to the underside of the massage table 106. According to one embodiment, the barrier device 300 further includes a fastener (not illustrated) at the bottom 306 of the closed sidewall 302 for attachment of the bottom 306 of underneath of a massage table 300. The attachment of the bottom 306 to the massage table 300 is configured to form a hollow cavity of the closed side wall 302.

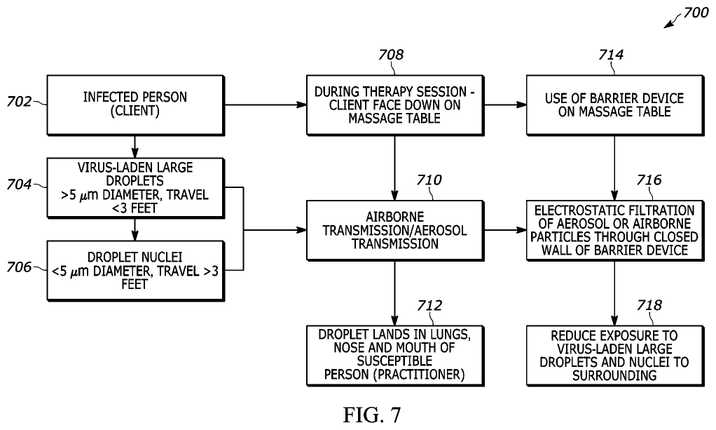

FIG. 7 is a flowchart illustrating a method of filtration of virus-laden large droplets during close proximity services sessions in accordance with some embodiments. A process of filtration virus-laden large droplets from the infected person (client) during close proximity services sessions will be described hereinafter with reference to FIG. 7 indicated as 700, beginning with operation 702. In operation 702, the infected person is responsible for production of virus-laden droplets. Virus-laden droplets are greater than 5 μm (micrometer) in diameter and may travel nearly three (3) feet in distance in the surrounding area as indicated in operation 704. In operation 706, viruses can still spread through aerosols, or tiny respiratory droplets called droplet nuclei that the infected person coughs or otherwise releases into the air.

According to the operation 708, the client faces down on a massage table which leads to aerosol particles transmission. Aerosol transmission is plausible when infectious aerosols are generated by or from an infectious person, the pathogen remains viable in the environment for some period of time, and the target tissues in which the pathogen initiates infection are accessible to the aerosol as illustrated in operations 710 and 712.

According to the operations 714 and 716, when the barrier device is installed on the massage table it acts as a filter for prevention of transmission of aerosol or airborne particles due its electrostatic mechanism and allows airborne particles to adhere on the surface of the barrier device. Absorption of airborne particles using the barrier device reduces the exposure of the aerosol particles in the surrounding.

According to some embodiments, the barrier device 300 may be formed of any shape or size. In an alternate embodiment, the barrier device 300 may be composed of additional or fewer components. In the one of the embodiment, the barrier device 300 is designed to cover an area of the massage table that is below the face cradle of the massage table in order to prevent release of infectious respiratory droplets in the surroundings as well as other exposed portions of the massage table and face cradle in that area.

According to an embodiment, the closed side wall 302 of the barrier device 300 may further include protective filters layer along with the washable product materials for prevention of release of air droplets in the surrounding.

As used herein, “airborne” refers to infectious agents that may be transmitted from persons or animals or environments by either “droplet infection” via the transmis-sion of infectious droplets, or by “airborne transmission” via the transmission of infectious “airborne droplet nuclei”. serious illnesses which are transmitted by airborne droplet nuclei include, but are not limited to, measles, varicella-zoster virus infections, legionella, disseminated zoster, tuberculosis, canine influenzas, psittacosis, inclusion body disease of boid snakes, foot and-mouth disease (FMD), avian influenzas including influenza A (H5N1), H5N9 and SARS-COVID-19.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element preceded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method for preventing the spread of a communicable disease, during a close proximity services session using a massage table incorporating a face cradle, the method comprising:
assembling a device to the face cradle prior to the close proximity services session; and
capturing, by the device, one or more respiratory droplets excreted by a client during the close proximity services session.

2. The method for preventing the spread of a communicable disease during a close proximity services session of claim 1, wherein the close proximity services comprise one or more selected from a group comprising massage therapy, medical examination, reflexology, physiotherapy, acupuncture, and reiki healing.

3. The method for preventing the spread of a communicable disease during a close proximity services session of claim 1, further comprising after the close proximity services session:
removing the device; and
cleaning the device.

4. The method for preventing the spread of a communicable disease during a close proximity services session of claim 3, further comprising:
repeating the assembling, capturing, removing, and cleaning operations for each of a plurality of close proximity services sessions.

5. The method for preventing the spread of a communicable disease during a close proximity services session of claim 1, wherein the capturing comprises:
operating as a filter for prevention of transmission of one or more particles using an electrostatic mechanism which allows airborne particles to adhere to a surface of the device.

6. The method for preventing the spread of a communicable disease during a close proximity services session of claim 5, wherein the one or more particles comprise one or more of an airborne particle and an aerosol particle.

7. The method for preventing the spread of a communicable disease during a close proximity services session of claim 1, wherein the device comprises a sidewall having a closed side and an open side, a top portion, and a bottom portion, wherein the top portion includes a securing means, and further wherein the assembling operation comprises:
securing the device to the face cradle using the securing means.

8. The method for preventing the spread of a communicable disease during a close proximity services session of claim 7, wherein the assembling operation further comprises:
attaching the bottom portion of the device to an underside of the massage table.

9. A device for preventing the spread of a communicable disease, during a close proximity services session using a massage table incorporating a face cradle, the device comprising:
a closed sidewall;
an open sidewall;
a top portion mechanically connected to the closed and to the open sidewall, wherein the top portion includes a securing means for securing the top portion to the face cradle; and
a bottom portion mechanically connected to the closed sidewall.

10. The device for preventing the spread of a communicable disease during a close proximity services session of claim 9, wherein the closed side wall and the open side wall runs vertically downwards underneath the face cradle.

11. The device for preventing the spread of a communicable disease during a close proximity services session of claim 9, wherein the closed sidewall and the open sidewall form a vertically cylindrical hammock shape.

12. The device for preventing the spread of a communicable disease during a close proximity services session of claim 9, wherein the bottom portion of the closed side wall further includes a fastener for attaching the bottom portion of the closed side wall to an underside of the massage table.

13. The device for preventing the spread of a communicable disease during a close proximity services session of claim 9, wherein the securing means comprises one or more of a hook-and-loop fastener, a snap closure, and an elastic element.

14. The device for preventing the spread of a communicable disease during a close proximity services session of claim 9, wherein the securing means provides easy access for removal of the device from the face cradle for each of plurality of close proximity services sessions.

15. The device for preventing the spread of a communicable disease during a close proximity services session of claim 9, wherein the closed side wall and the top portion of the closed side wall is constructed from a single or multilayer washable product materials selected from a group of cotton, polyester, copper fabric, quilting fabric and silk.

16. The device for preventing the spread of a communicable disease during a close proximity services session of claim 9, wherein the close proximity services comprise one or more selected from a group comprising massage therapy, medical examination, reflexology, physiotherapy, acupuncture, and reiki healing.

17. The device for preventing the spread of a communicable disease during a close proximity services session of claim 9, wherein the device operates as a collector to capture one or more respiratory droplets excreted from a client.

18. The device for preventing the spread of a communicable disease during a close proximity services session of claim 9, wherein the device operates as a filter for prevention of transmission of one or more particles using an electrostatic mechanism which allows airborne particles to adhere to a surface of the device.

19. The device for preventing the spread of a communicable disease during a close proximity services session of claim 9, wherein the open sidewall maintains an air flow in the device.
